(19) United States
(12) Patent Application Publication
(10) Pub. No.: US 2020/0237804 A1
(43) Pub. Date: Jul. 30, 2020

(54) METHODS AND COMPOSITIONS FOR TREATING CHRONIC RHINOSINUSITIS

(71) Applicant: GLYCOMIRA THERAPEUTICS, INC., Salt Lake City, UT (US)

(72) Inventors: Glenn D. PRESTWICH, Spokane, WA (US); Abigail PULSIPHER, Salt Lake City, UT (US); Thomas P. KENNEDY, Charlotte, NC (US); Jeremiah Andrew ALT, Salt Lake City, UT (US)

(21) Appl. No.: 16/610,936

(22) PCT Filed: Oct. 18, 2018
(86) PCT No.: PCT/US2018/056419

§ 371 Date: Nov. 5, 2019

Related U.S. Application Data
(60) Provisional application No. 62/573,903, filed on Oct. 18, 2017.

(51) Int. Cl. A61K 31/728; A61K 31/737; A61L 33/08

(52) U.S. Cl. CPC A61K 31/728; A61K 9/0043; A61L 33/08; A61K 31/737

(57) ABSTRACT

Described herein is the use of a methylated/sulfated hyaluronan, sulfated hyaluronan, or the pharmaceutically acceptable salt or ester thereof for the treatment of chronic rhinosinusitis.

### CROSS REFERENCE TO RELATED APPLICATIONS

This application claims priority upon U.S. provisional application Ser. No. 62/573,903, filed Oct. 18, 2017. This application is hereby incorporated by reference in its entirety.

### ACKNOWLEDGMENTS

This invention was made with government support under Grant R43AI126987 awarded by the National Institute of Allergy and Infectious Diseases and Grant KL2TR001065 awarded by National Center for Advancing Translational Sciences. The government has certain rights in the invention.

### BACKGROUND

Chronic rhinosinusitis (CRS) is a debilitating condition of sinonasal inflammation that affects up to 16% of the U.S. population.^{1,2} Patients with CRS experience significant declines in quality of life, with associated comorbidities including depression, migraines, cognitive deficits, and sleep dysfunction.^{3,5} These comorbidities contribute to a phenotype that is more crippling than life-threatening conditions such as end-stage renal disease and coronary artery disease.^{6,7} The annual expenditure to treat patients with CRS is $64B, accounting for 5% of the total U.S. health care budget, with an additional estimated cost of $13B attributed to lost work productivity.⁸⁻¹⁰ Despite its wide prevalence, financial and societal burden, and effect on quality of life, CRS remains an under-researched epidemic with limited effective treatment options.^{11,12}

### SUMMARY

Described herein is the use of a methylated/sulfated hyaluronan, sulfated hyaluronan, or the pharmaceutically acceptable salt or ester thereof for the treatment of chronic rhinosinusitis. The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows the study design to examine the anti-inflammatory properties of a methylated/sulfated hyaluronan in a murine model of chronic rhinosinusitis.

FIG. 2A shows GM-1111 significantly reduces the number of recorded clinical signs in mice given intranasal A. fumigatus. FIG. 2B shows treatment with GM-1111 induces similar weight growth trends to those of the healthy controls.

FIG. 3 shows GM-1111 reduces A. fumigatus-induced inflammation in the sinuses of mice. Microscopic images of sinonasal tissues stained with hematoxylin and eosin show coronal sections (2×) and respective higher magnification images (20×) of the indicated region of the respiratory (box) and olfactory (circle) tissue. The sinonasal tissues from A. fumigatus-treated animals demonstrate degenerative changes in all epithelial layers (arrows), marked inflammatory cell infiltration, and thickening in the respiratory mucosa (star). These changes were less pronounced in animals treated with GM-1111.

FIG. 4 shows GM-1111 reduces A. fumigatus-induced changes in the sinuses of mice. Microscopic images of sinonasal olfactory tissues stained with Alcian Blue (mucopolysaccharides) and Nuclear Fast Red (nuclei). The sinonasal tissues from A. fumigatus-treated animals demonstrate increased goblet cell hyperplasia (arrows) tissue remodeling (PCNA, brown signal). These changes were much less pronounced after treatment with GM-1111.

FIG. 5A shows mice treated with A. fumigatus demonstrated a significant increase in blood eosinophils (% of total white blood cells), whereas GM-1111 treatment showed a reduction. FIG. 5B shows animals treated with A. fumigatus demonstrated a significant increase in CD4+ cell infiltration, and treatment with GM-1111 showed a significant reduction compared to disease controls.

FIGS. 6A and 6B show GM-1111 significantly reduces A. fumigatus-induced increases in (A) serum IgE levels and (B) gene expression of inflammatory tissue cytokines common in human CRS. The genes were normalized to housekeeping genes and plotted as the gene expression level relative to healthy controls (dotted line).

FIGS. 7A and 7B show GM-1111 suppresses the growth and biofilm formation of opportunistic pathogens common in CRS. FIG. 7A shows the flow cytometry data of overnight broth culture counts in the presence of GM-1111. Data are expressed as the mean±SD. FIG. 7B shows the scanning electron microscopic images showing the reduction of S. aureus counts and biofilm when incubated with 0.5% GM-1111 for 36 hours.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optional bioactive agent” means that the bioactive agent may or may not be present.

Throughout this specification, unless the context dictates otherwise, the word “comprise,” or variations such as “comprises” or “comprising,” will be understood to imply the inclusion of a stated element, integer, step, or group of elements, integers, or steps, but not the exclusion of any other element, integer, step, or group of elements, integers, or steps.

The term “treat” as used herein is defined as maintaining or reducing the symptoms of a pre-existing condition when compared to the same condition in the absence of the methylated/sulfated hyaluronan. The term “prevent” as used herein is defined as eliminating or reducing the likelihood of the occurrence of one or more symptoms of a disease or disorder when compared to the same symptom in the absence of the methylated/sulfated hyaluronan. The term “inhibit” as used herein is the ability of the compounds described herein to completely eliminate the activity or reduce the activity when compared to the same activity in the absence of the methylated/sulfated hyaluronan.

“Subject” refers to mammals including, but not limited to, humans, non-human primates, sheep, dogs, rodents (e.g., mouse, rat, etc.), guinea pigs, cats, rabbits, cows, and non-mammals including chickens, amphibians, and reptiles.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

As used herein, a plurality of items, structural elements, compositional elements, and/or materials may be presented in a common list for convenience. However, these lists should be construed as though each member of the list is individually identified as a separate and unique member. Thus, no individual member of any such list should be construed as a de facto equivalent of any other member of the same list based solely on its presentation in a common group, without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range was explicitly recited. As an example, a numerical range of “about 1” to “about 5” should be interpreted to include not only the explicitly recited values of about 1 to about 5, but also to include individual values and sub-ranges within the indicated range. Thus, included in this numerical range are individual values such as 2, 3, and 4, the sub-ranges such as from 1-3, from 2-4, from 3-5, from about 1-about 3, from 1 to about 3, from about 1 to 3, etc., as well as 1, 2, 3, 4, and 5, individually. The same principle applies to ranges reciting only one numerical value as a minimum or maximum. Furthermore, such an interpretation should apply regardless of the breadth or range of the characters being described.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc., of these materials are disclosed, that while specific reference of each various individual and collective combination and permutation of these compounds may not be explicitly disclosed, each is specifically contemplated and described herein. For example, if a class of molecules A, B, and C are disclosed, as well as a class of molecules D, E, and F, and an example of a combination A+D is disclosed, then even if each is not individually recited, each is individually and collectively contemplated. Thus, in this example, each of the combinations A+E, A+F, B+D, B+E, B+F, C+D, C+E, and C+F, are specifically contemplated and should be considered disclosed from disclosure of A, B, and C; D, E, and F; and the example combination of A+D. Likewise, any subset or combination of these is also specifically contemplated and disclosed. Thus, for example, the sub-group of A+E, B+F, and C+E is specifically contemplated and should be considered disclosed from disclosure of A, B, and C; D, E, and F; and the example combination of A+D. This concept applies to all aspects of this disclosure including, but not limited to, steps in methods of making and using the disclosed compositions. Thus, if there exist a variety of additional steps that can be performed with any specific embodiment or combination of embodiments of the disclosed methods, each such combination is specifically contemplated and should be considered disclosed.

Described herein is the use of a methylated/sulfated hyaluronan, sulfated hyaluronan, or the pharmaceutically acceptable salt or ester thereof for the treatment of chronic rhinosinusitis.

In one aspect, at least one primary C-6 hydroxyl proton of the N-acetyl-glucosamine residue of hyaluronan is substituted with a methyl group. In other aspects, the amount of base is sufficient to deprotonate from 0.001% to 100%, 1% to 100% 5% to 100%, 10% to 100%, 20% to 100%, 50% to 100%, 60% to 100%, 70% to 100%, 80% to 100%, 90% to 100%, or 95% to 100% of the primary C-6 hydroxyl protons of the N-acetyl-glucosamine residue of the hyaluronan starting material or derivative thereof are replaced with a methyl group.

The degree of sulfation of the methylated/sulfated hyaluronan or sulfated hyaluronan can vary from partial sulfation to complete sulfation. In general, free hydroxyl groups not methylated can be sulfated. In one aspect, at least one C-2 hydroxyl proton and/or C-3 hydroxyl proton is substituted with a sulfate group. In another aspect, the degree of sulfation is from 0.5, 1.0, 1.5, 2.0, 2.5, 3.0, 3.5, or less than 4.0 or any range thereof (e.g., 2.5 to 3.5, 3.0 to 4.0, etc.) per disaccharide unit of the methylated/sulfated hyaluronan. In one aspect, the amount of base is sufficient to deprotonate from 0.001% to 100%, 1% to 100% 5% to 100%, 10% to 100%, 20% to 100%, 50% to 100%, 60% to 100%, 70% to 100%, 80% to 100%, 90% to 100%, or 95% to 100% of the primary C-6 hydroxyl protons of the N-acetyl-glucosamine residue of the hyaluronan starting material or derivative thereof are replaced with a sulfate group.

The molecular weight of the methylated/sulfated hyaluronan or sulfated hyaluronan can vary depending upon reaction conditions. In one aspect, the average molecular weight of the methylated/sulfated hyaluronan or sulfated hyaluronan is from 1 kDa to 50 kDa, 1 kDa to 25 kDa, 1 kDa to 20 kDa, 1 kDa to 15 kDa, 1 kDa to 10 kDa, 1 kDa to 9 kDa, 1 kDa to 8 kDa, or 2 kDa to 7 kDa, 3 kDa to 7 kDa, 4 kDa to 7 kDa, 4 kDa to 6 kDa, or 5 kDa to 6 kDa.

In one aspect, when the sulfating agent is a pyridine-sulfur trioxide complex, a pyridinium adduct of the methylated/sulfated hyaluronan or sulfated hyaluronan is produced, where pyridine is covalently attached to the partially or fully sulfated hyaluronan. Not wishing to be bound by theory, when hyaluronan is reacted with the pyridine-sulfur trioxide complex in a solvent such as, for example, DMF, a small amount of acid is produced from traces of water present in situ, which causes partial depolymerization resulting in a free reducing end group. The hydroxyl group of the hemiketal can ultimately be sulfated to produce a sulfated intermediate, which subsequently reacts with free pyridine produced in situ to produce the pyridinium adduct.

In one aspect, the methylated/sulfated hyaluronan has the formula depicted below:

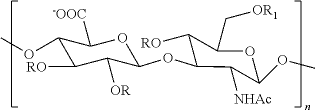

In this aspect, R₁ is a methyl group, while the remaining R groups are sulfate groups alone or in combination with hydrogen. In one aspect, the n is from 5 to 20, 5 to 15, 5 to 10, or 7 to 9.

In another aspect, a mixture composed of a first methylated/sulfated hyaluronan and a second methylated/sulfated hyaluronan with pyridine covalently bonded to the methylated/sulfated hyaluronan can be used in the methods described herein. In one aspect, the mixture includes
(a) a first modified hyaluronan or a pharmaceutically acceptable salt or ester thereof, wherein said first modified hyaluronan or its pharmaceutically acceptable salt or ester comprises (i) at least one primary C-6 hydroxyl proton of at least one N-acetyl-glucosamine residue substituted with a methyl group, (ii) an average molecular weight from 1 kDa to 15 kDa, (iii) a degree of methylation greater than 0 to 0.5 methyl groups per disaccharide unit; and (iv) a degree of sulfation of 2.5 to 4.0 sulfate groups per disaccharide unit; and
(b) a second modified hyaluronan or a pharmaceutically acceptable salt or ester thereof, wherein said second modified hyaluronan or its pharmaceutically acceptable salt or ester comprises (i) at least one primary C-6 hydroxyl proton of at least one N-acetyl-glucosamine residue substituted with a methyl group, (ii) an average molecular weight from 1 kDa to 15 kDa, (iii) a degree of methylation greater than 0 to 0.5 methyl groups per disaccharide unit; and a (iv) degree of sulfation of 2.5 to 4.0 sulfate groups per disaccharide unit, wherein pyridine is covalently bonded to the second modified hyaluronan or a pharmaceutically acceptable salt or ester thereof.

In one aspect, the degree of methylation in the first and second modified hyaluronan is 0.030, 0.050, 0.075, 0.100, 0.125, 0.150, 0.175, 0.200, 0.225, 0.250, 0.275, 0.300, 0.325, 0.350, 0.375, 0.400, 0.425, 0.45, 0.475, or 0.500 methyl groups per disaccharide unit, where any value can be a lower and upper endpoint of a range (e.g., 0.030 to 0.300, 0.100 to 0.200, etc.). In one aspect, only the primary C-6 hydroxyl proton of an N-acetyl-glucosamine residue of the first and second modified hyaluronan is substituted with the methyl group (i.e., methyl group is only at this position). In other aspects, 1% to 100% 5% to 100%, 10% to 100%, 20% to 100%, 50% to 100%, 60% to 100%, 70% to 100%, 80% to 100%, 90% to 100%, or 95% to 100% of the primary C-6 hydroxyl protons of the N-acetyl-glucosamine residue of the first and second modified hyaluronan are replaced with a methyl group.

In another aspect, the first and second modified hyaluronan have an average molecular weight 1 kDa, 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa, or 15 kDa, where any value can be a lower and upper endpoint of a range (e.g., 1 kDa to 10 kDa, 3 kDa to 7 kDa, etc.).

In another aspect, the first and second modified hyaluronan have a degree of sulfation of 2.5, 2.75, 3.00, 3.25, 3.50, 3.75, or 4.00 sulfate groups per disaccharide unit, where any value can be a lower and upper endpoint of a range (e.g., 1.5 to 3.5, 3. to 4.0, etc.).

In another aspect, the amount of pyridine in the mixture of the first and second modified hyaluronan is 0.10, 0.25, 0.50, 0.75, 1.00, 1.25, 1.50, 1.75, 2.00, 2.25, 2.50, 2.75, 3.00, 3.25, 3.50, 3.75, or 4.00 wt % of the mixture, where any value can be a lower and upper endpoint of a range (e.g., 0.500 to 3.00, 1.00 to 2.00, etc.). The amount of pyridine can be quantified by ¹H NMR and UV spectroscopy.

In another aspect, the degree of methylation in the first and second modified hyaluronan is 0.03 to 0.3 methyl groups per disaccharide unit, the first and second modified hyaluronan has an average molecular weight from 1 kDa to 10 kDa, the degree of sulfation in the first and second modified hyaluronan is 3.0 to 4.0 sulfate groups per disaccharide unit, and the amount of pyridine present in the composition is from 0.1 wt % to 4.0 wt % of the composition.

The methylated/sulfated hyaluronan or sulfated hyaluronan useful herein can be the pharmaceutically acceptable salt or ester thereof. In some aspects, the pharmaceutically acceptable ester can be a prodrug. For example, free hydroxyl groups of the methylated/sulfated hyaluronan or sulfated hyaluronan can be partially esterified with palmitoyl chloride to afford an amphiphilic compound that is hydrolyzed by endogenous esterases to liberate the methylated/sulfated hyaluronan or sulfated hyaluronan. Other prosthetic groups that liberate non-toxic byproducts familiar to those skilled in the art may also be used. Pharmaceutically acceptable salts are prepared by treating the free acid with an appropriate amount of a pharmaceutically acceptable base. Representative pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, benzalkonium, choline, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. The molar ratio of the methylated/sulfated hyaluronan to base used is chosen to provide the ratio desired for any particular salt. For preparing, for example, the ammonium salts of the free acid starting material, the starting material can be treated with approximately one equivalent of pharmaceutically acceptable base to yield a neutral salt. In other aspects, choline salts of the methylated/sulfated hyaluronan or sulfated hyaluronan can be prepared and used herein.

The methylated/sulfated hyaluronan, sulfated hyaluronan, or salt/ester thereof can be formulated in any excipient to produce pharmaceutical compositions for intranasal administration. Examples of such excipients include, but are not limited to, water, aqueous hyaluronic acid, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol. In certain aspects, the pH can be modified depending upon the mode of administration. For example, the pH of the composition is from about 5 to about 6, which is suitable for topical applications. Additionally, the pharmaceutical compositions can include carriers, thickeners, diluents, preservatives, surface active agents and the like in addition to the compounds described herein.

In one aspect, the methylated/sulfated hyaluronan or sulfated hyaluronan is formulated as a spray, wash, lavage, or other suitable formulations typically used in nasal applications.

In certain aspects, the methylated/sulfated hyaluronan or sulfated hyaluronan can be formulated with one or more bioactive agents that are used to treat sinus inflammation. For example, the methylated/sulfated hyaluronan or sulfated hyaluronan can be formulated with steroid sprays (e.g., Flonase®, Nasacort®, Nasonex®).

The pharmaceutical compositions can be prepared using techniques known in the art. In one aspect, the composition is prepared by admixing the methylated/sulfated hyaluronan or sulfated hyaluronan with a pharmaceutically-acceptable compound and/or carrier. The term “admixing” is defined as mixing the two components together so that there is no chemical reaction or physical interaction. The term “admixing” also includes the chemical reaction or physical interaction between the compound and the pharmaceutically-acceptable compound. Covalent bonding to reactive therapeutic drugs, e.g., those having nucleophilic groups, can be undertaken on the compound. Second, non-covalent entrapment of a pharmacologically active agent in a cross-linked polysaccharide is also possible. Third, electrostatic or hydrophobic interactions can facilitate retention of a pharmaceutically-acceptable compound in the compounds described herein.

It will be appreciated that the actual preferred amounts of the methylated/sulfated hyaluronan or sulfated hyaluronan in a specified case will vary according to the specific compound being utilized, the particular compositions formulated, the mode of application, and the particular situs and subject being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (1999).

In one aspect, the dosage of the methylated/sulfated hyaluronan is less than 1,000 μg per unit dose. In another aspect, the dosage of the methylated/sulfated hyaluronan, sulfated hyaluronan, or the salt/ester thereof is from 100 ng to 1,000 μg, 200 ng to 1,000 μg, 300 ng to 1,000 μg, 400 ng to 1,000 μg, 500 ng to 1,000 μg, 500 ng to 900 μg, 500 ng to 800 μg, 500 ng to 700 μg, 500 ng to 600 μg, 500 ng to 500 μg, 500 ng to 400 μg, 500 ng to 300 μg per, or 500 ng to 200 μg unit dose. The methylated/sulfated hyaluronan, sulfated hyaluronan, or the salt/ester thereof can be administered once a day or multiple times per day as needed. In other aspects, the methylated/sulfated hyaluronan, sulfated hyaluronan, or the salt/ester thereof can be administered two or more days as needed.

The methylated/sulfated hyaluronan, sulfated hyaluronan, or the salt/ester thereof described herein is useful in treating chronic rhinosinusitis (CRS). The pathophysiology of CRS encompasses a wide range of inflammatory profiles, and therefore CRS management necessitates multiple therapies to target its multi-factorial etiology.² Not wishing to be bound by theory, the pathological characteristics of CRS include: (1) migration and infiltration of innate and adaptive immune cells into the sinonasal tissue, (2) increased permeability and damage to the sinonasal epithelial cell barrier, and (3) decreased mucociliary clearance and mucus accumulation with increased susceptibility to bacterial infection.^{14,41} Recent therapeutic attempts have been directed to classify CRS into two primary inflammatory clusters: Th1- and Th2-driven inflammation.

The Examples below demonstrate that methylated/sulfated hyaluronan can be used to treat CRS. The methylated/sulfated hyaluronan is highly water soluble and can be readily formulated in physiological buffers for increased sinonasal epithelial and mucosal penetration,¹⁸ a key advantage over nasal steroid sprays, which demonstrate less than 3% distribution and penetration within the sinuses.⁵⁰

Not wishing to be bound by theory, the methylated/sulfated hyaluronan, sulfated hyaluronan, or the salt/ester thereof inhibits multiple inflammatory mediators while specifically targeting early inflammatory signaling. In one aspect, the methylated/sulfated hyaluronan, sulfated hyaluronan, or the salt/ester thereof reduces inflammatory cell migration and invasion into the sinonasal mucosa and epithelium, resulting in the local reduction of cytokine gene expression.

In another aspect, the methylated/sulfated hyaluronan, sulfated hyaluronan, or the salt/ester thereof can treat or prevent one or more rhinologic symptoms of chronic rhinosinusitis such as, for example, nasal erythema, nasal congestion, rhinorrhea, reduction or loss of the sense of smell, itchy nose, sneezing, difficulty in breathing, eating, and drinking, or any combination thereof.

CRS is clinically characterized by sinonasal inflammation with olfactory and respiratory epithelial breakdown, mucosal thickening, goblet cell hyperplasia, and increased inflammatory cell infiltration.^{14,41} In one aspect, the methylated/sulfated, sulfated hyaluronan, or the salt/ester thereof hyaluronan can reduce degenerative changes to the olfactory and respiratory epithelium, tissue thickening, and goblet cell hyperplasia. This is demonstrated below in the Examples.

The severity of sinonasal inflammation and success of a therapeutic intervention can be determined by quantifying the involvement of key immune cells and inflammatory biomarkers of CRS in tissues. For example, the abundance of eosinophils in the whole blood of animals as a percent of total white blood cells can be used to evaluate the degree of inflammation, where an increase in eosinophils is indicative of sinonasal inflammation. In one aspect, the methylated/sulfated hyaluronan, sulfated hyaluronan, or the salt/ester thereof can reduce the presence or amount of eosinophils present in a subject that has CRS. This is demonstrated in the Examples, where it was demonstrated in vivo that the methylated/sulfated hyaluronan reduced A. fumigatus-induced increases in blood eosinophil counts and CD4+ cell infiltration into sinonasal tissues.

CRS is a complex condition with multiple etiologies and subtypes that are characterized by unique or mixed inflammatory profiles. In one aspect, the methylated/sulfated hyaluronan, sulfated hyaluronan, or the salt/ester thereof can reduce serum IgE protein levels, which is associated with inflammatory genes. This is demonstrated in the Examples, where it is shown in vivo that methylated/sulfated hyaluronan reduced A. fumigatus-induced increases in serum IgE levels and gene expression of inflammatory tissue cytokines common in human CRS.

In another aspect, the methylated/sulfated hyaluronan, sulfated hyaluronan, or the salt/ester thereof can reduce anosmia or dysnosmia

In another aspect, the methylated/sulfated hyaluronan, sulfated hyaluronan, or the salt/ester thereof can reduce bacterial growth and biofilm formation in a subject. Not wishing to be bound by theory, the microbiome in the upper airway is critical to maintain homeostasis. As such, bacteria have a symbiotic relationship and are universally present in the sinuses of patients with CRS. Rather than serving a primary infectious role, evidence suggests that pathogenic bacterial colonization and biofilm formation occur when the air-mucosal barrier breaks down due to chronic inflammatory signaling. Over one-third of patients with CRS are indirectly infected with biofilm-forming bacteria, contributing to recalcitrant CRS.^{55,56} Moreover, early inflammatory signaling such as that mediated through TLR2 complicates the severity of inflammatory response that is thought to lead to impaired mucocilliary clearance and ostial obstruction, altering the normal bacterial homeostasis and creating an environment more susceptible to opportunistic pathogens.⁵⁷

### Examples

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Methods

### Study Compounds

A. fumigatus extracts were obtained from Stallergenes-Greer Laboratories (Lenoir, N.C.).

The methylated/sulfated hyaluronan (referred to below as GM-1111) was synthesized using the following procedures.

Preparation of Low Molecular Weight Hyaluronan
1. Slowly dissolve 20 g of 850 kDa HA (1% w/v) into 1.7 L of ddH₂O while vigorously stirring over heat (˜40° C.). When all 20 g of HA is added, remove from heat and stir until cooled to room temperature, then slowly add 333 mL 6N HCl while stirring. Stir at room temperature for approximately 2 weeks.
2. Use HPLC, GPC, or SEC to monitor degradation reaction at 14 days. Neutralize each sample before analysis to stop reaction and analysis by UV detection at 232 nm, comparing to previous batches of methylated/sulfated hyaluronan.
3. At the molecular weight range of 3-5 kDa, neutralize the reaction to pH 7.0 by slowly adding 40% (w/v) NaOH over ice.
4. Dialyze in 1000 MWCO dialysis tubing against ddH₂O for 24 hrs, changing the water every 6 hrs to obtain hyaluronan fragments of greater than 1 kDa.
5. Lyophilize to obtain a white, fluffy solid. Yield: 12.032 g, 60.2%

Preparation of Methylated Hyaluronan
1. Dissolve 6.0 g (4% w/v HA in NaOH solution) of low molecular weight hyaluronan in 150 mL of a 40% w/v solution of NaOH in ddH₂O, and stir the mixture for 2 hours at room temperature, which generates a viscous solution.
2. Add 225 mL of isopropanol and continue stirring.
3. Add 6 mL (6 eq) of iodomethane, and stir the mixture for 24 hours at room temperature.
4. After 24 hours, use a separation funnel to remove the organic solvent layer from the viscous aqueous layer, and add 300 mL of ddH₂O to dilute the crude methylated hyaluronan.
5. Adjust the solution to pH 7.0 with 6N HCl on ice.
6. Allow the neutralized solution to warm to room temperature, and add 3 L of MeOH:EtOH (1:2 v/v) while stirring to precipitate the methylated hyaluronan intermediate. Collect the product by filtration, and dry it in a vacuum oven.

Sulfation of Methylated Hyaluronan to Produce GM-1111
1. Add 2.5 g of crude methylated hyaluronan to 200 mL of anhydrous DMF and stir for 1 h prior to adding 1.56 mL of tributylamine (1 eq). Stir the solution for 20 min at room temperature.
2. Add 25 g of pyridine-sulfur trioxide (24 eq.) by adding 5 g at a time.
3. Stir the mixture for 3 h at 40° C.
4. Cool the reaction on ice, and add 50 mL of ddH₂O to quench the reaction.
5. Precipitate the crude material by adding 250 mL of cold 95% ethanol saturated with anhydrous sodium acetate.
6. Centrifuge the crude product at 4,500 rpm for 5-10 min, and decant the liquid to collect the light brown gummy solid.
7. Dissolve the crude product in ddH₂O, and dialyze against 20 L of 100 mM NaCl, changing the solution four times a day over 24 h, followed by dialysis against 20 L of distilled water 4 times over 24 h.
8. Lyophilize the dialyzed material. Final Yield: 42.0% of methylated/sulfated hyaluronan (GM-1111)
9. The methylated/sulfated hyaluronan had the following characteristics: average molecular weight is 3 kDa to 7 kDa; average methyl groups per disaccharide unit is 0.3 to 0.3; average degree of sulfation of 3.0 to 4.0; and average pyridine content is 0.1 to 4.0 wt % (pyridine content used in experiments below is 0.69 wt %).

### Animals

Male BALB/c mice (8-10 weeks old) were purchased from Charles River Laboratories (Santa Clara, Calif.) and housed in pathogen-free conditions at the University of Utah's Comparative Medicine Center. Procedures were performed under the regulation of the Institutional Animal Care and Use Committee (IACUC) at the University of Utah (15-11021) and according to the Guide for the Care and Use of Laboratory Animals.

### Animal Model

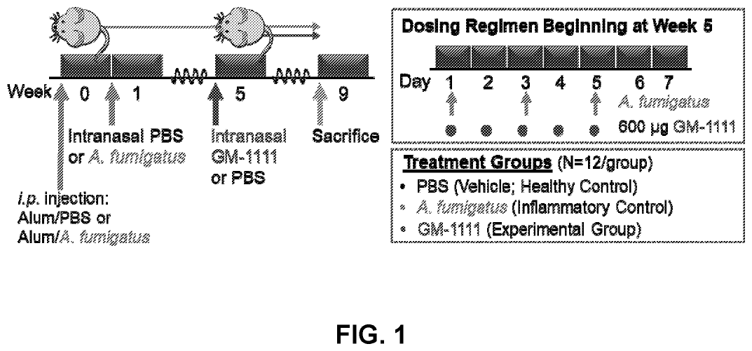

The animal model timeline, dosing regimen, and treatment groups are illustrated in FIG. 1. The following study groups were used: PBS (vehicle; healthy control, N=12), A. fumigatus+PBS (inflammatory control, N=12), and A. fumigatus+GM-1111 (experimental group, N=12). The PBS group was sensitized with an intraperitoneal (i.p.) injection of 200 μL of PBS/Imject™ Alum Adjuvant (1:1 solution) (ThermoFisher Scientific, Pittsburgh, Pa.), whereas the A. fumigatus+PBS and A. fumigatus+GM-1111 groups received 200 μL of 20,000 PNU/mL A. fumigatus extracts/Imject Alum Adjuvant. After 1 week, the animals were intranasally administered 10 μL of PBS (Sigma Aldrich, St. Louis, Mo.) or A. fumigatus extracts (20,000 PNU/mL PBS) per nare 3× weekly for 4 weeks. This regiment is well known to generate significant chronic sinonasal mucosa inflammation.⁴⁰ Intranasal treatment of GM-1111 in PBS (300 μg dose/nare, 5× weekly) or PBS (10 μL) began at week 5 and was continued for 4 weeks. A. fumigatus extract administration (3× weekly) was continued during treatment to maintain a high level of inflammation. At week 9, whole blood was collected, and the animals were sacrificed and examined for histologic changes and inflammatory tissue biomarkers associated with CRS. Body weight measurements and behavioral (clinical) signs (e.g., nasal erythema, scratching nose, sneezing, and holding breath/gasping) were recorded 3× weekly throughout the study.

### Tissue Processing

All study animals were sacrificed at week 9, and tissues were processed for histological, immunohistochemical, and biochemical analyses. Animals were placed under heavy anesthesia through isoflurane and sacrificed via exsanguination and cervical dislocation. Sinonasal tissue was harvested and placed in 4% formalin (Ted Pella, Redding, Calif.) for 48 hours. The tissues were subsequently decalcified using 14% ethylenediaminetetraacetic acid (EDTA, pH 7.2) (Sigma Aldrich, St. Louis, Mo.) for 2 weeks, followed by coronal sectioning of sinonasal tissues under an Olympus FSX100 stereoscope (Olympus Inc., Center Valley, Pa.). Coronal sections were cut (4 μm), paraffin-embedded, slide-mounted, and stained with hematoxylin and eosin (H&E) or left unstained for further analyses by HistoTox Labs (Boulder, Colo.).

### Blood Eosinophil Quantification

Whole blood was collected in EDTA-coated microcentrifuge tubes at the time of sacrifice and subjected to complete blood count, differential smear, and manual white blood cell (WBC) differential analyses, performed by SRI Biosciences (Menlo Park, Calif.).

### Immunohistochemical (IHC) and Staining Analyses

Sinonasal tissues were deparaffinized in xylene (3×10 min) and rehydrated using decreasing concentrations of ethanol (100%, 95%, and 70%, 2×5 min) and ddH₂O (2×5 min). Unless stated, all staining reagents were obtained from and used as recommended by Vector Laboratories (Burlingame, Calif.).

Acid Mucopolysaccharides (Goblet Cells) and Dividing Cells (Tissue Remodeling):

Tissues were stained using a NovaUltra™ Alcian Blue/Nuclear Fast Red Solution Staining Kit (IHC World, Woodstock, Md.) following the supplier's instructions and then subjected to staining for proliferating cell nuclear antigen (PCNA). Antigen retrieval was performed in citrate buffer (pH 6.0), and tissues were blocked in BLOXALL and then subjected to IHC detection of mouse anti-mouse PCNA (1:6000) (Abcam, Cambridge, Mass.) using Mouse on Mouse (M.O.M.™) and ImmPACT DAB Peroxidase Kits.

T Cells:

Antigen retrieval was performed in Tris-OH buffer (pH 8.0), and tissues were blocked in BLOXALL and then subjected to IHC detection of rabbit anti-mouse CD4 (1:1000) (Abcam, Cambridge, Mass.) using ImmPRESS™ HRP anti-rabbit IgG and ImmPACT DAB Peroxidase Kits. Tissues were imaged under an Olympus BX43 upright microscope (Olympus Inc., Pittsburgh, Pa.) using an EOS Rebel T2i digital SLR camera (Canon Inc., Melville, N.Y.). The severity of CD4+ cell infiltration was determined by counting the number of CD4+ cells present in the olfactory and respiratory epithelium and mucosa in a similar coronal section for each animal and assigning a severity index of 0 (no), 1 (focal), 2 (mild), 3 (moderate), or 4 (severe) with respect to the number/presence of CD4+ cells.

### IgE Expression Quantification

Total serum IgE was determined using an ELISA MAX™ Deluxe Mouse IgE Kit (Biolegend, San Diego, Calif.) following the manufacturer's instructions. IgE concentration was determined from a standard curve and normalized to the total protein in each sample, reported as nanogram per milligram of total serum protein.

### Gene Expression Profiling

After cutting and slide-mounting for histological and IHC analyses, sinonasal tissues embedded in paraffin were subjected to paraffin tissue punching (olfactory epithelial and mucosal tissue), nucleic acid extraction, and gene expression analyses using Inflammation V2 gene panels (NanoString Technologies, Seattle, Wash.), which were performed by the Biorepository and Molecular Pathology Core and the Molecular Diagnostic Core at the Huntsman Cancer Institute (University of Utah, Salt Lake City, Utah). Gene expression levels were normalized to five housekeeping genes and analyzed using NSolver® Software (NanoString Technologies, Seattle, Wash.). The data are reported as the fold change relative to healthy controls (PBS group).

### Statistical Analysis

Statistical analyses were performed using Prism 6 for Windows (GraphPad Software; La Jolla, Calif.). Pair-wise comparisons were made by one-way ANOVA, followed by Tukey's post hoc test to adjust for multiple comparisons (p value <0.05 indicates a statistically significant difference).

### Results

Murine Clinical Signs and Body Weight Observations.

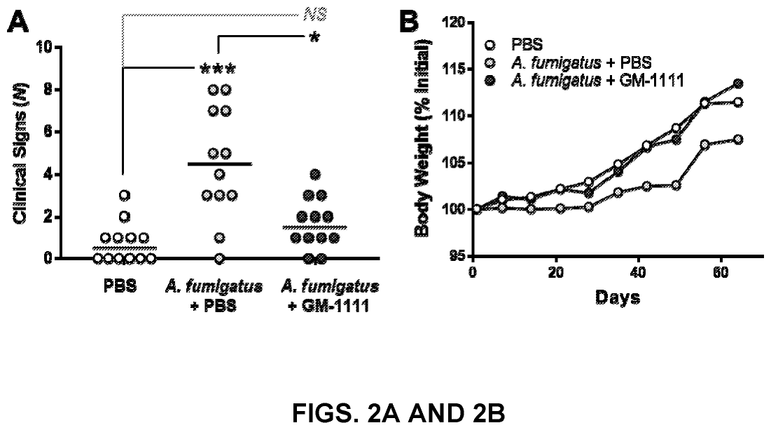

Clinical signs and body weight measurements were recorded throughout the development and treatment of the model. Observations indicating sinus irritation were noted by the appearance of the nose (edema and erythema) and sneezing, whereas nasal congestion was characterized by gasping and holding of breath. Compared to healthy controls, there was a significant increase in the number of recorded clinical signs in the disease controls (p<0.001; FIG. 2A) and a significant decrease in overall growth (p<0.01; FIG. 2B), expressed as the percent increase of initial body weight (100%). By contrast, treatment with GM-1111 significantly reduced the clinical signs observed in mice when compared to disease controls (p<0.05). The average body weight of GM-1111-treated animals showed a similar growth trend to that of healthy controls (10-12% increase, p<0.01), which was significant when compared to the growth trend of the A. fumigatus group (p<0.01).

Inflammation-Induced Damages to the Sinonasal Tissues.

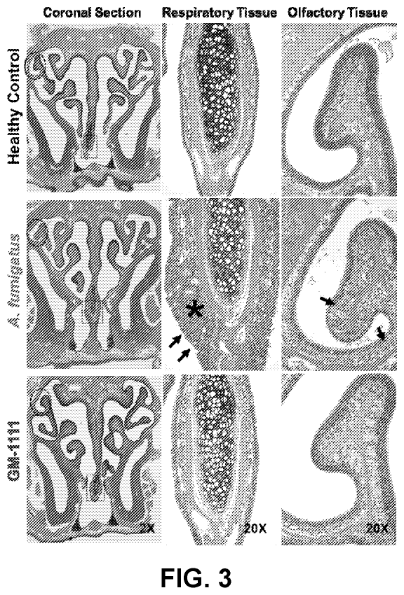

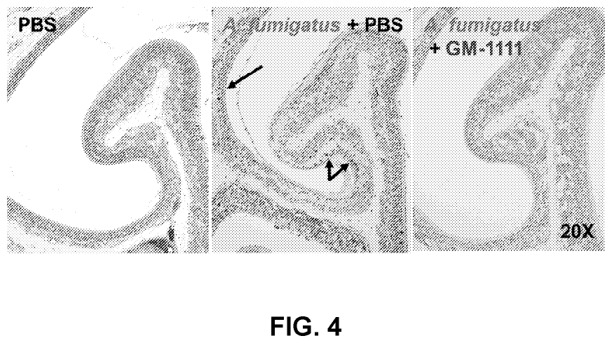

CRS is clinically characterized by sinonasal inflammation with olfactory and respiratory epithelial breakdown, mucosal thickening, goblet cell hyperplasia, and increased inflammatory cell infiltration.^{14,41} FIG. 3 demonstrates different tissue sections composed of respiratory and olfactory epithelium and mucosa to highlight the global tissue damage with A. fumigatus administration and the effects of GM-1111 to reduce this damage. Compared to the sinonasal tissues from healthy controls, tissues from the disease group were histologically characterized by degenerative changes in all epithelial layers (arrows), marked inflammatory cell infiltration, generalized thickening in the respiratory epithelium (star), and increased goblet cell hyperplasia (arrows, FIG. 4). Similar changes were also observed in the olfactory epithelium with atrophied olfactory epithelial layers (arrows, FIG. 3) and increased inflammatory cell infiltration. These changes were also accompanied by a global increase in tissue remodeling, as demonstrated by elevated levels of proliferating cell nuclear antigen (PCNA, brown signal) expressed by dividing cells (FIG. 4).⁴² By contrast, the sinonasal tissues treated with GM-1111 demonstrated reduced degenerative changes to the olfactory and respiratory epithelium, tissue thickening, and goblet cell hyperplasia, as well as similar levels of tissue regeneration (PCNA) to those of healthy controls (FIGS. 3 and 4).

Analyses of Tissue Biomarkers.

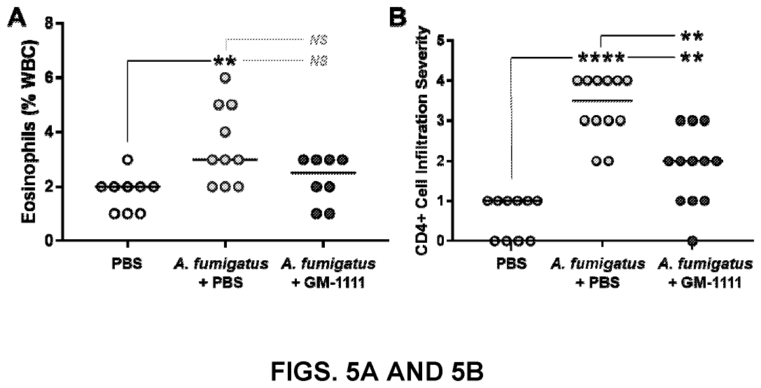

The severity of sinonasal inflammation and success of a therapeutic intervention can be determined by quantifying the involvement of key immune cells and inflammatory biomarkers of CRS in tissues. The abundance of eosinophils in the whole blood of animals was quantified as a percent of total white blood cells. Compared to healthy controls, there was a significant increase in eosinophil numbers in the blood collected from the disease group (p<0.01; FIG. 5A). In contrast, GM-1111-treated animals demonstrated a reduction (not significant) in blood eosinophils.

The histological data demonstrated increased inflammatory cell infiltration in the sinonasal tissues harvested from the disease group compared to controls. T cell infiltration is characteristic of all CRS subtypes, therefore the severity of T cell infiltration was counted and scored by immunohistochemical analysis of CD4+ immune cells in sinonasal tissues of all animals.^{43,44} Compared to healthy controls, a significant increase with a median of ‘moderate to severe’ CD4+ cell infiltration was measured for the disease group (p<0.0001, FIG. 5B). Although there was also a significant increase in CD4+ cell infiltration in tissues from GM-1111-treated animals, CD4+ cell infiltration was significantly reduced compared to the disease group (p<0.01).

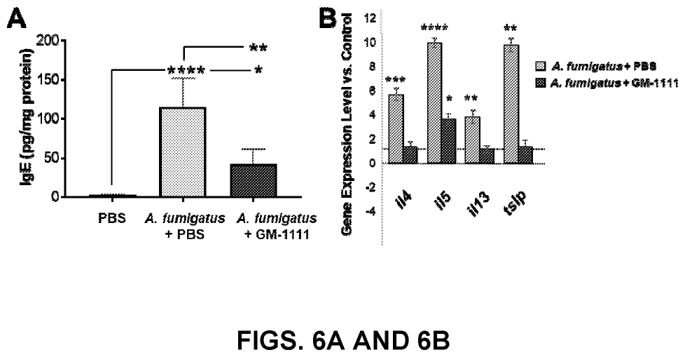

CRS is a complex condition with multiple etiologies and subtypes that are characterized by unique or mixed inflammatory profiles. The expression levels of key inflammatory genes associated with human CRS with respect to inflammatory profile and serum IgE were quantified. Consistent with human CRS and reports using the A. fumigatus mouse model, significant increases in serum IgE protein levels were measured in mice treated with allergen vs. controls (p<0.0001, FIG. 6A).⁴⁵⁻⁴⁷ A significant 2.7-fold reduction in IgE was measured in animals treated with GM-1111 (p<0.05). Similarly, significant increases, ranging from 4 to 10-fold, in tslp, il4, il5 and il13 expression were measured compared to healthy controls (p<0.0001 to 0.05, FIG. 6B). Expression of these genes was significantly reduced, most of which was driven back to baseline, with GM-1111 treatment.

GM-1111 Suppresses Bacterial Growth and Disrupts Biofilm Formation.

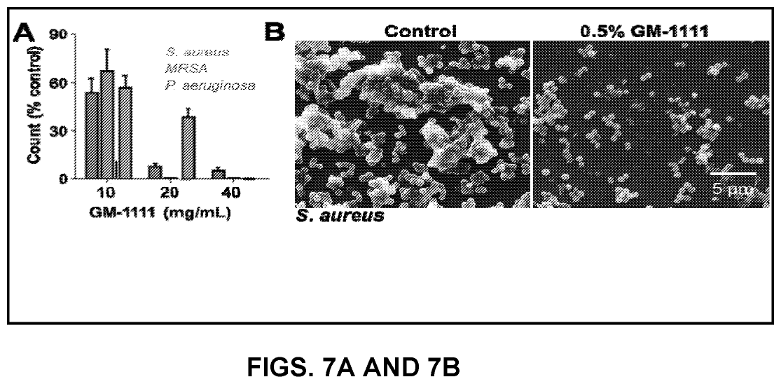

GM-1111 suppresses both Gram-positive and Gram-negative bacterial growth (FIG. 7A; 20 mg/mL) and disrupts biofilm formation of S. aureus (5 mg/mL) (FIG. 7B).

### REFERENCES

1. Bachert C, Pawankar R, Zhang L et al. ICON: chronic rhinosinusitis. World Allergy Organ J 2014; 7:25.
2. Orlandi R R, Kingdom T T, Hwang P H et al. International Consensus Statement on Allergy and Rhinology: Rhinosinusitis. Int Forum Allergy Rhinol 2016; 6 Suppl 1:S22-209.
3. Alt J A, Mace J C, Buniel M C, Soler Z M, Smith T L. Predictors of olfactory dysfunction in rhinosinusitis using the brief smell identification test. The Laryngoscope 2014.
4. Schlosser R J, Gage S E, Kohli P, Soler Z M. Burden of illness: A systematic review of depression in chronic rhinosinusitis. Am J Rhinol Allergy 2016; 30:250-256.
5. Alt J A, DeConde A S, Mace J C, Steele T O, Orlandi R R, Smith T L. Quality of Life in Patients With Chronic Rhinosinusitis and Sleep Dysfunction Undergoing Endoscopic Sinus Surgery: A Pilot Investigation of Comorbid Obstructive Sleep Apnea. JAMA otolaryngology—head & neck surgery 2015:1-9.
6. DeConde A S, Soler Z M. Chronic rhinosinusitis: Epidemiology and burden of disease. Am J Rhinol Allergy 2016; 30:134-139.
7. Soler Z M, Wittenberg E, Schlosser R J, Mace J C, Smith T L. Health state utility values in patients undergoing endoscopic sinus surgery. Laryngoscope 2011; 121:2672-2678.
8. Bhattacharyya N. Incremental health care utilization and expenditures for chronic rhinosinusitis in the United States. Ann Otol Rhinol Laryngol 2011; 120:423-427.
9. Bhattacharyya N. Functional limitations and workdays lost associated with chronic rhinosinusitis and allergic rhinitis. Am J Rhinol Allergy 2012; 26:120-122.
10. Caulley L, Thavorn K, Rudmik L, Cameron C, Kilty S J. Direct costs of adult chronic rhinosinusitis by using 4 methods of estimation: Results of the US Medical Expenditure Panel Survey. J Allergy Clin Immunol 2015; 136:1517-1522.
11. Rudmik L. Chronic rhinosinusitis: an under-researched epidemic. J Otolaryngol Head Neck Surg 2015; 44:11.
12. Tan B K, Kern R C, Schleimer R P, Schwartz B S. Chronic rhinosinusitis: the unrecognized epidemic. Am J Respir Crit Care Med 2013; 188:1275-1277.
13. Kennedy J L, Borish L. Chronic sinusitis pathophysiology: the role of allergy. Am J Rhinol Allergy 2013; 27:367-371.
14. Schleimer R P. Immunopathogenesis of Chronic Rhinosinusitis and Nasal Polyposis. Annu Rev Pathol 2017; 12:331-357.
15. Tomassen P, Vandeplas G, Van Zele T et al. Inflammatory endotypes of chronic rhinosinusitis based on cluster analysis of biomarkers. J Allergy Clin Immunol 2016; 137:1449-1456 e1444.
16. Lee S, Lane A P. Chronic rhinosinusitis as a multifactorial inflammatory disorder. Curr Infect Dis Rep 2011; 13:159-168.
17. Hamilos D L. Chronic rhinosinusitis: epidemiology and medical management. J Allergy Clin Immunol 2011; 128:693-707; quiz 708-699.
18. Rudmik L, Hoy M, Schlosser R J et al. Topical therapies in the management of chronic rhinosinusitis: an evidence-based review with recommendations. Int Forum Allergy Rhinol 2013; 3:281-298.
19. Orlandi R R, Kingdom T T, Hwang P H. International Consensus Statement on Allergy and Rhinology: Rhinosinusitis Executive Summary. International forum of allergy & rhinology 2016; 6 Suppl 1:S3-21.
20. Rudmik L, Schlosser R J, Smith T L, Soler Z M. Impact of topical nasal steroid therapy on symptoms of nasal polyposis: a meta-analysis. Laryngoscope 2012; 122:1431-1437.
21. Schwartz J S, Tajudeen B A, Cohen N A. Medical management of chronic rhinosinusitis—an update. Expert Rev Clin Pharmacol 2016; 9:695-704.
22. Snidvongs K, Thanaviratananich S. Update on Intranasal Medications in Rhinosinusitis. Curr Allergy Asthma Rep 2017; 17:47.
23. Smith K A, Rudmik L. Medical therapy, refractory chronic rhinosinusitis, and productivity costs. Curr Opin Allergy Clin Immunol 2017; 17:5-11.
24. Bhattacharyya N. Ambulatory sinus and nasal surgery in the United States: demographics and perioperative outcomes. Laryngoscope 2010; 120:635-638.
25. Smith T L, Litvack J R, Hwang P H et al. Determinants of outcomes of sinus surgery: a multi-institutional prospective cohort study. Otolaryngol Head Neck Surg 2010; 142:55-63.
26. Bachert C, Gevaert P, Hellings P. Biotherapeutics in Chronic Rhinosinusitis with and without Nasal Polyps. J Allergy Clin Immunol Pract 2017.
27. Souza-Fernandes A B, Pelosi P, Rocco P R. Bench-to-bedside review: the role of glycosaminoglycans in respiratory disease. Crit Care 2006; 10:237.
28. Abbadi A, Lauer M, Swaidani S, Wang A, Hascall V. Hyaluronan Rafts on Airway Epithelial Cells. J Biol Chem 2016; 291:1448-1455.
29. Manzanares D, Monzon M E, Savani R C, Salathe M. Apical oxidative hyaluronan degradation stimulates airway ciliary beating via RHAMM and RON. Am J Respir Cell Mol Biol 2007; 37:160-168.
30. Turino G M, Cantor J O. Hyaluronan in respiratory injury and repair. Am J Respir Crit Care Med 2003; 167:1169-1175.
31. Yildiz-Pekoz A, Ozsoy Y. Inhaled Heparin: Therapeutic Efficacy and Recent Formulations. J Aerosol Med Pulm Drug Deliv 2017; 30:143-156.
32. Johnson Z, Proudfoot A E, Handel T M. Interaction of chemokines and glycosaminoglycans: a new twist in the regulation of chemokine function with opportunities for therapeutic intervention. Cytokine Growth Factor Rev 2005; 16:625-636.
33. Ledson M, Gallagher M, Hart C A, Walshaw M. Nebulized heparin in Burkholderia cepacia colonized adult cystic fibrosis patients. Eur Respir J 2001; 17:36-38.
34. Rao N V, Argyle B, Xu X et al. Low anticoagulant heparin targets multiple sites of inflammation, suppresses heparin-induced thrombocytopenia, and inhibits interaction of RAGE with its ligands. Am J Physiol Cell Physiol 2010; 299:C97-110.
35. Pulsipher A, Qin X, Thomas A J, Prestwich G D, Oottamasathien S, Alt J A. Prevention of sinonasal inflammation by a synthetic glycosaminoglycan. Int Forum Allergy Rhinol 2017; 7:177-184.
36. Oottamasathien S, Jia W, McCoard L et al. A murine model of inflammatory bladder disease: cathelicidin peptide induced bladder inflammation and treatment with sulfated polysaccharides. J Urol 2011; 186:1684-1692.
37. Savage J R, Pulsipher A, Rao N V et al. A Modified Glycosaminoglycan, GM-0111, Inhibits Molecular Signaling Involved in Periodontitis. PLoS One 2016; 11:e0157310.
38. Zhang J, Xu X, Rao N V et al. Novel sulfated polysaccharides disrupt cathelicidins, inhibit RAGE and reduce cutaneous inflammation in a mouse model of rosacea. PLoS One 2011; 6:e16658.
39. Prestwich G, Zhang, J, Rao, N V, Xu, X, Kennedy, T P. Alkylated Semi-synthetic Glycosaminoglycan Ethers, and Methods of Making and of Use Thereof. International Published Patent Application No. WO2009/124266.
40. Lindsay R, Slaughter T, Britton-Webb J et al. Development of a murine model of chronic rhinosinusitis. Otolaryngol Head Neck Surg 2006; 134:724-730; discussion 731-722.
41. Stevens W W, Lee R J, Schleimer R P, Cohen N A. Chronic rhinosinusitis pathogenesis. J Allergy Clin Immunol 2015; 136:1442-1453.
42. Nakagawa T, Yamane H, Nakai Y, Shigeta T, Takashima T, Takeda Z. Comparative assessment of cell proliferation and accumulation of extracellular matrix in nasal polyps. Acta Otolaryngol Suppl 1998; 538:205-208.
43. Derycke L, Eyerich S, Van Crombruggen K et al. Mixed T helper cell signatures in chronic rhinosinusitis with and without polyps. PLoS One 2014; 9:e97581.
44. Pant H, Hughes A, Schembri M, Miljkovic D, Krumbiegel D. CD4(+) and CD8(+) regulatory T cells in chronic rhinosinusitis mucosa. Am J Rhinol Allergy 2014; 28:e83-89.
45. Baba S, Kondo K, Suzukawa M, Ohta K, Yamasoba T. Distribution, subtype population, and IgE positivity of mast cells in chronic rhinosinusitis with nasal polyps. Ann Allergy Asthma Immunol 2017.
46. Pakdaman M N, Corry D B, Luong A. Fungi linking the pathophysiology of chronic rhinosinusitis with nasal polyps and allergic asthma. Immunol Invest 2011; 40:767-785.
47. Schuh J M, Hoselton S A. An inhalation model of allergic fungal asthma: Aspergillus fumigatus-induced inflammation and remodeling in allergic airway disease. Methods Mol Biol 2013; 1032:173-184.
48. Hascall V, Esko J D. Hyaluronan. In: Varki A, Cummings R D, Esko J D, Stanley P, Hart G W, Aebi M, Darvill A G, Kinoshita T, Packer N H, Prestegard J H, Schnaar R L, Seeberger P H, eds. Essentials of Glycobiology. Cold Spring Harbor (N.Y.), 2015.
49. Jura-Szoltys E, Chudek J. Epistaxis as the reason for premature discontinuation of clopidogrel after percutaneous coronary angioplasty with stent implantation. Kardiol Pol 2011; 69:817-823.
50. Liang J, Lane A P. Topical Drug Delivery for Chronic Rhinosinusitis. Curr Otorhinolaryngol Rep 2013; 1:51-60.
51. Jia M, Chen Z, Du X, Guo Y, Sun T, Zhao X. A simple animal model of Staphylococcus aureus biofilm in sinusitis. Am J Rhinol Allergy 2014; 28:e115-119.
52. Liu T, Kong W, Yang P, Wang B. A possible association of Staphylococcus enterotoxin B-induced asthma and sinusitis. J Huazhong Univ Sci Technolog Med Sci 2006; 26:63-67.
53. Jacob A, Chole R A. Survey anatomy of the paranasal sinuses in the normal mouse. Laryngoscope 2006; 116:558-563.
54. Treuting P, Dintzis, S M. Comparative Anatomy and Histology: A Mouse and Human Atlas. Elsevier Inc., 2012.
55. Al-Mutairi D, Kilty S J. Bacterial biofilms and the pathophysiology of chronic rhinosinusitis. Curr Opin Allergy Clin Immunol 2011; 11:18-23.
56. Kilty S J, Desrosiers M Y. The role of bacterial biofilms and the pathophysiology of chronic rhinosinusitis. Curr Allergy Asthma Rep 2008; 8:227-233.
57. Sun Y, Zhou B, Wang C et al. Biofilm formation and Toll-like receptor 2, Toll-like receptor 4, and NF-kappaB expression in sinus tissues of patients with chronic rhinosinusitis. Am J Rhinol Allergy 2012; 26:104-109.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1-30. (canceled)

31. A method for treating chronic rhinosinusitis in a subject comprising nasally administering to the subject in need of treatment a composition comprising
(a) a first modified hyaluronan or a pharmaceutically acceptable salt or ester thereof, wherein said first modified hyaluronan or its pharmaceutically acceptable salt or ester comprises (i) an average molecular weight from 2 kDa to 8 kDa, (ii) a degree of methylation greater than 0 to 0.5 at the primary C-6 hydroxyl group of the N-acetyl-glucosamine residue of the first modified hyaluronan; and (iii) a degree of sulfation of 2.5 to 4.0 sulfate groups per disaccharide unit; and
(b) a second modified hyaluronan or a pharmaceutically acceptable salt or ester thereof, wherein said second modified hyaluronan or its pharmaceutically acceptable salt or ester comprises (i) an average molecular weight from 2 kDa to 8 kDa, (ii) a degree of methylation greater than 0 to 0.5 at the primary C-6 hydroxyl group of the N-acetyl-glucosamine residue of the first modified hyaluronan; and a (iii) degree of sulfation of 2.5 to 4.0 sulfate groups per disaccharide unit, wherein pyridine is covalently bonded to the second modified hyaluronan or a pharmaceutically acceptable salt or ester thereof,
wherein the composition reduces gene expression, of interleukin-1-beta (Il1b), interleukin-4 (Il4), interleukin-5 (Il5), interleukin-6 (Il6), interleukin-13 (Il13), interleukin-17-alpha (Il17a), interleukin-22 (Il22), interferon-gamma (Ifng), transforming growth factor-beta (Tgfb), thymic stromal lymphopoietin (Ts1p), tumor necrosis factor-alpha (Tnfa), or any combination thereof, wherein the gene expression is reduced in the subject compared to the same subject not administered the composition.

32. The method of claim 31, wherein the composition reduces gene expression, of interleukin-1-beta (Il1b), interleukin-4 (Il4), interleukin-5 (Il5), interleukin-6 (Il6), interleukin-13 (Il13), interleukin-17-alpha (Il17a), interleukin-22 (Il22), interferon-gamma (Ifng), transforming growth factor-beta (Tgfb), thymic stromal lymphopoietin (Ts1p), and tumor necrosis factor-alpha (Tnfa).

33. The method of claim 31, wherein the gene is interleukin-4 (Il4), interleukin-5 (Il5), and interleukin-13 (Il13).

34. The method of claim 31, wherein the gene is interleukin-17-alpha (Il17a) and interferon-gamma (Ifng).

35. The method of claim 31, wherein the gene is interleukin-1-beta (Il1b), interleukin-6 (Il6), and tumor necrosis factor-alpha (Tnfa).

36. The method of claim 31, wherein the degree of methylation in the first and second modified hyaluronan is 0.03 to 0.3 at the primary C-6 hydroxyl group of the N-acetyl-glucosamine residue of the first and second modified hyaluronan.

37. The method of claim 31, wherein the degree of sulfation in the first and second modified hyaluronan is 3.0 to 4.0 sulfate groups per disaccharide unit.

38. The method of claim 31, wherein the amount of pyridine present in the composition is from 0.1 wt % to 4.0 wt % of the composition.

39. The method of claim 31, the degree of methylation in the first and second modified hyaluronan is 0.03 to 0.3 at the primary C-6 hydroxyl group of the N-acetyl-glucosamine residue of the first and second modified hyaluronan, the first and second modified hyaluronan has an average molecular weight from 2 kDa to 8 kDa, the degree of sulfation in the first and second modified hyaluronan is 3.0 to 4.0 sulfate groups per disaccharide unit, and the amount of pyridine present in the composition is from 0.1 wt % to 4.0 wt % of the composition.

40. The method of claim 31, wherein the pharmaceutically acceptable ester of the first and second modified hyaluronan is a prodrug.

41. The method of claim 31, wherein the composition is administered as a spray, aerosol, nasal wash, or lavage.

42. The method of claim 31, wherein the pharmaceutically acceptable salt of the first and second modified hyaluronan comprises an organic salt, a metal salt, or a combination thereof.

43. The method of claim 31, wherein the pharmaceutically acceptable salt of the first and second modified hyaluronan is a salt of NH₄⁺, Na⁺, Li⁺, K⁺, Ca⁺², Mg⁺², Fe⁺², Fe⁺³, Cu⁺², Al⁺³, Zn⁺², 2-trimethylethanolammonium cation (choline), or a quaternary salt of isopropylamine, trimethylamine, diethylamine, triethylamine, tripropylamine, ethanolamine, 2-dimethylaminoethanol, 2-diethylaminoethanol, lysine, arginine, or histidine.

44. The method of claim 31, wherein the composition treats or prevents one or more rhinologic symptoms of chronic rhinosinusitis.

45. The method of claim 44, wherein the symptom is nasal erythema, nasal congestion, rhinorrhea, reduction or loss of the sense of smell, itchy nose, sneezing, difficulty in breathing, eating, and drinking, or any combination thereof.

46. The method of claim 31, wherein the composition reduces degenerative changes to the olfactory and respiratory epithelium, tissue thickening, goblet cell hyperplasia, or any combination thereof in the subject compared to the same subject not administered the composition.

47. The method of claim 31, wherein the composition reduces the amount of eosinophils in the subject compared to the same subject not administered the composition.

48. The method of claim 31, wherein the composition reduces the amount of serum IgE protein levels in the subject compared to the same subject not administered the composition.

49. The method of claim 31, wherein the composition treats or prevents anosmia or dysnosmia in the subject compared to the same subject not administered the composition.

50. The method of claim 31, wherein the composition inhibits or prevents bacterial growth and biofilm formation in the subject compared to the same subject not administered the composition.

51. A method for reducing the expression of a gene in a subject comprising administering to the subject a composition comprising
(a) a first modified hyaluronan or a pharmaceutically acceptable salt or ester thereof, wherein said first modified hyaluronan or its pharmaceutically acceptable salt or ester comprises (i) an average molecular weight from 2 kDa to 8 kDa, (ii) a degree of methylation greater than 0 to 0.5 at the primary C-6 hydroxyl group of the N-acetyl-glucosamine residue of the first modified hyaluronan; and (iii) a degree of sulfation of 2.5 to 4.0 sulfate groups per disaccharide unit; and
(b) a second modified hyaluronan or a pharmaceutically acceptable salt or ester thereof, wherein said second modified hyaluronan or its pharmaceutically acceptable salt or ester comprises (i) an average molecular weight from 2 kDa to 8 kDa, (ii) a degree of methylation greater than 0 to 0.5 at the primary C-6 hydroxyl group of the N-acetyl-glucosamine residue of the first modified hyaluronan; and a (iii) degree of sulfation of 2.5 to 4.0 sulfate groups per disaccharide unit, wherein pyridine is covalently bonded to the second modified hyaluronan or a pharmaceutically acceptable salt or ester thereof,
wherein the gene is interleukin-1-beta (Il1b), interleukin-4 (Il4), interleukin-5 (Il5), interleukin-6 (Il6), interleukin-13 (Il13), interleukin-17-alpha (Il17a), interleukin-22 (Il22), interferon-gamma (Ifng), transforming growth factor-beta (Tgfb), thymic stromal lymphopoietin (Ts1p), tumor necrosis factor-alpha (Tnfa), or any combination thereof, wherein the expression of the gene in the subject is reduced compared to the same subject not administered the composition.

52. The method of claim 51, wherein the gene is interleukin-1-beta (Il1b), interleukin-4 (Il4), interleukin-5 (Il5), interleukin-6 (Il6), interleukin-13 (Il13), interleukin-17-alpha (Il17a), interleukin-22 (Il22), interferon-gamma (Ifng), transforming growth factor-beta (Tgfb), thymic stromal lymphopoietin (Ts1p), and tumor necrosis factor-alpha (Tnfa).

53. The method of claim 51, wherein the gene is interleukin-4 (Il4), interleukin-5 (Il5), and interleukin-13 (Il13).

54. The method of claim 51, wherein the gene is interleukin-17-alpha (Il17a) and interferon-gamma (Ifng).

55. The method of claim 51, wherein the gene is interleukin-1-beta (Il1b), interleukin-6 (Il6), and tumor necrosis factor-alpha (Tnfa).

56. The method of claim 51, wherein the degree of methylation in the first and second modified hyaluronan is 0.03 to 0.3 at the primary C-6 hydroxyl group of the N-acetyl-glucosamine residue of the first and second modified hyaluronan.

57. The method of claim 51, wherein the degree of sulfation in the first and second modified hyaluronan is 3.0 to 4.0 sulfate groups per disaccharide unit.

58. The method of claim 51, wherein the amount of pyridine present in the composition is from 0.1 wt % to 4.0 wt % of the composition.

59. The method of claim 51, wherein the degree of methylation in the first and second modified hyaluronan is 0.03 to 0.3 at the primary C-6 hydroxyl group of the N-acetyl-glucosamine residue of the first and second modified hyaluronan, the first and second modified hyaluronan has an average molecular weight from 2 kDa to 8 kDa, the degree of sulfation in the first and second modified hyaluronan is 3.0 to 4.0 sulfate groups per disaccharide unit, and the amount of pyridine present in the composition is from 0.1 wt % to 4.0 wt % of the composition.

60. The method of claim 51, wherein the pharmaceutically acceptable ester of the first and second modified hyaluronan is a prodrug.

61. The method of claim 51, wherein the composition is administered as a spray, aerosol, nasal wash, lavage, or any combination thereof.

62. The method of claim 51, wherein the pharmaceutically acceptable salt of the first and second modified hyaluronan comprises an organic salt, a metal salt, or a combination thereof.

63. The method of claim 51, wherein the pharmaceutically acceptable salt of the first and second modified hyaluronan is a salt of NH₄⁺, Na⁺, Li⁺, K⁺, Ca⁺², Mg⁺², Fe⁺², Fe⁺³, Cu⁺², Al⁺³, Zn⁺², 2-trimethylethanolammonium cation (choline), or a quaternary salt of isopropylamine, trimethylamine, diethylamine, triethylamine, tripropylamine, ethanolamine, 2-dimethylaminoethanol, 2-diethylaminoethanol, lysine, arginine, or histidine.

64. The method of claim 51, wherein the composition is nasally administered to the subject.

65. A method for reducing serum IgE protein levels in a subject comprising administering to the subject a composition comprising
(a) a first modified hyaluronan or a pharmaceutically acceptable salt or ester thereof, wherein said first modified hyaluronan or its pharmaceutically acceptable salt or ester comprises (i) an average molecular weight from 2 kDa to 8 kDa, (ii) a degree of methylation greater than 0 to 0.5 at the primary C-6 hydroxyl group of the N-acetyl-glucosamine residue of the first modified hyaluronan; and (iii) a degree of sulfation of 2.5 to 4.0 sulfate groups per disaccharide unit; and
(b) a second modified hyaluronan or a pharmaceutically acceptable salt or ester thereof, wherein said second modified hyaluronan or its pharmaceutically acceptable salt or ester comprises (i) an average molecular weight from 2 kDa to 8 kDa, (ii) a degree of methylation greater than 0 to 0.5 at the primary C-6 hydroxyl group of the N-acetyl-glucosamine residue of the first modified hyaluronan; and a (iii) degree of sulfation of 2.5 to 4.0 sulfate groups per disaccharide unit, wherein pyridine is covalently bonded to the second modified hyaluronan or a pharmaceutically acceptable salt or ester thereof,
wherein the amount of serum IgE protein levels in the subject is lower compared to the same subject not administered the composition.
